(19) United States
(12) Patent Application Publication
(10) Pub. No.: US 2025/0114604 A1
(43) Pub. Date: Apr. 10, 2025

(54) METHOD AND APPARATUS FOR PERSONALIZING NEUROSTIMULATION

(71) Applicant: Boston Scientific Neuromodulation Corporation, Valencia, CA (US)

(72) Inventors: Tianhe Zhang, Studio City, CA (US); Bradon Thio, Durham, NC (US)

(21) Appl. No.: 18/893,268

(22) Filed: Sep. 23, 2024

Related U.S. Application Data
(60) Provisional application No. 63/543,133, filed on Oct. 9, 2023.
(60) Provisional application No. 63/543,137, filed on Oct. 9, 2023.

(51) Int. Cl. A61N 1/36; A61N 1/372

(52) U.S. Cl. CPC A61N 1/36132; A61N 1/36139; A61N 1/37223; A61N 1/37247

(57) ABSTRACT

A system for delivering neurostimulation to a patient may include a personalization circuit and a pattern optimization circuit. The personalization circuit may include an assessment input to receive patient-specific information resulting from a patient survey, a calibration input configured to receive calibration information resulting from a calibration, and personalization processing circuitry. The patient-specific information may include patient dimensions of the neurostimulation. The calibration information may include the patient's response to the neurostimulation. The personalization processing circuitry may be configured to determine a personalized objective function using the patient dimensions and to determine a personalized model having a model state personalized to the patient's response to the neurostimulation. The pattern optimization circuit may be configured to determine optimal pattern(s) of neurostimulation using the personalized objective function and the personalized model for programming a stimulation device to deliver the neurostimulation according to the optimal pattern(s) of neurostimulation.

### CLAIM OR PRIORITY

This application claims the benefit of U.S. Provisional Application Nos. 63/543,133 and 63/543,137, both filed on Oct. 9, 2023, which are hereby incorporated by reference in their entireties.

### CROSS REFERENCE TO RELATED APPLICATION

This application is related to commonly assigned U.S. Provisional Patent Application Ser. No.______(Attorney Docket No. 6279.380US1), entitled “METHOD AND APPARATUS FOR OPTIMIZING NEUROSTIMULATION FOR A PATIENT”, filed on even date herewith, which is incorporated by reference herein in its entirety.

### TECHNICAL FIELD

This document relates generally to neurostimulation and more particularly to a method and system for personalization of neurostimulation therapy for a patient.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered to a patient in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. When neurostimulation is delivered to the patient for treating a medical condition (e.g., pain), the stimulation parameters used by a neurostimulator need to be determined based on capability and programmability of the neurostimulator, the patient's symptoms and preferences, and the patient's responses to the neurostimulation, among other factors and considerations.

### SUMMARY

An example (“Example 1”) of a system for delivering neurostimulation to a patient using a stimulation device is provided. The system may include a personalization circuit and a pattern optimization circuit. The personalization circuit may include an assessment input, a calibration input, and personalization processing circuitry. The assessment input may be configured to receive patient-specific information resulting from a patient survey. The patient-specific information may include one or more patient dimensions of the neurostimulation. The calibration input may be configured to receive calibration information resulting from a calibration. The calibration information may include the patient's response to the neurostimulation. The personalization processing circuitry may be configured to determine a personalized objective function using the one or more patient dimensions and to determine a personalized model having a model state personalized to the patient's response to the neurostimulation. The pattern optimization circuit may include optimization processing circuitry configured to determine one or more optimal patterns of neurostimulation using the personalized objective function and the personalized model for programming the stimulation device to deliver the neurostimulation according to the one or more optimal patterns of neurostimulation.

In Example 2, the subject matter of Example 1 may optionally be configured such that the personalization processing circuitry is configured to determine a personalized cost function as the personalized objective function. The personalized cost function is a quantitative measure of a condition of the patient expressed as a function of the one or more patient dimensions.

In Example 3, the subject matter of Example 2 may optionally be configured such that the personalization processing circuitry is configured to optimize the personalized model by minimizing a difference between the patient's response to the neurostimulation as predicted by simulation using the personalized model and the received patient's response to the neurostimulation.

In Example 4, the subject matter of any one or any combination of Examples 1 to 3 may optionally be configured such that the personalization processing circuitry is configured to select the one or more optimal patterns of neurostimulation from stored patterns of neurostimulation.

In Example 5, the subject matter of any one or any combination of Examples 1 to 4 may optionally be configured such that the personalization processing circuitry is configured to determine the personalized model by selecting a model state from stored predefined model states.

In Example 6, the subject matter of any one or any combination of Examples 1 to 4 may optionally be configured such that the personalization processing circuitry is configured to determine the personalized model by adjusting parameters of a stored model.

In Example 7, the subject matter of any one or any combination of Examples 1 to 6 may optionally be configured to include an implantable stimulator as the stimulation device and an external system communicatively coupled to the implantable stimulator via telemetry. The external system includes the personalization circuit and the pattern optimization circuit.

In Example 8, the subject matter of Example 7 may optionally be configured such that the external system includes an external programmer including the pattern optimization circuit and a remote device communicatively coupled to the external programmer via a telecommunication network and including the personalization circuit.

In Example 9, the subject matter of Example 7 may optionally be configured such that the external system includes an external programmer including the personalization circuit and the pattern optimization circuit.

In Example 10, the subject matter of any one or any combination of Examples 7 to 9 may optionally be configured such that the external system is configured to perform the patient assessment including conducting a patient survey to determine the one or more patient dimensions of the neurostimulation.

In Example 11, the subject matter of any one or any combination of Examples 7 to 10 may optionally be configured such that the external system is configured to perform the calibration to determine the patient's response to the neurostimulation by programming the implantable stimulator to deliver the neurostimulation to the patient and measuring one or more parameters representing the patient's response.

In Example 12, the subject matter of Example 11 may optionally be configured such that the external system is configured to produce one or more response curves each being a measured parameter of the measured one or more parameters plotted as a function of a stimulation parameter used by the implantable stimulator to control the delivery of the neurostimulation.

An example (“Example 13”) of a non-transitory computer-readable storage medium including instructions is also provided. The instructions, which when executed by a system, cause the system to perform a method for delivering neurostimulation to a patient using a stimulation device. The method may include receiving patient-specific information resulting from a patient survey and receiving calibration information resulting from a calibration. The patient-specific information may include one or more patient dimensions of the neurostimulation. The calibration information may include the patient's response to the neurostimulation. The method may further include determining a personalized objective function using the received one or more patient dimensions, determining a personalized model having a model state personalized to the patient's response to the neurostimulation, determining one or more optimal patterns of neurostimulation using the personalized objective function and the personalized model, and generating information for programming the stimulation device to deliver the neurostimulation according to the one or more optimal patterns of neurostimulation.

In Example 14, the subject matter of determining the personalized objective function as found in Example 13 may optionally include determining a personalized cost function being a quantitative measure of a condition of the patient expressed as a function of the one or more patient dimensions.

In Example 15, the subject matter of any one or a combination of Examples 13 and 14 may optionally further include optimizing the personalized model by matching the patient's response to the neurostimulation as predicted by simulation using the personalized model to the received patient's response to the neurostimulation.

An example (“Example 16”) of a method for delivering neurostimulation to a patient is also provided. The method may include receiving patient-specific information resulting from a patient survey and receiving calibration information resulting from a calibration. The patient-specific information may include one or more patient dimensions of the neurostimulation. The calibration information may include the patient's response to the neurostimulation. The method may further include determining a personalized objective function using the received one or more patient dimensions, determining a personalized model having a model state personalized to the patient's response to the neurostimulation, determining one or more optimal patterns of neurostimulation using the personalized objective function and the personalized model, and programming a stimulation device to deliver the neurostimulation according to the one or more optimal patterns of neurostimulation.

In Example 17, the subject matter of determining the personalized objective function as found in Example 16 may optionally include determining a personalized cost function being a quantitative measure of a condition of the patient expressed as a function of the one or more patient dimensions.

In Example 18, the subject matter of any one or a combination of Examples 16 and 17 may optionally include optimizing the personalized model by matching the patient's response to the neurostimulation as predicted by simulation using the personalized model to the received patient's response to the neurostimulation.

In Example 19, the subject matter of determining the one or more optimal patterns of neurostimulation as found in any one or any combination of Examples 16 to 18 may optionally include selecting the one or more optimal patterns of neurostimulation from predefined patterns of neurostimulation.

In Example 20, the subject matter of determining the personalized model as found in any one or any combination of Examples 16 to 19 may optionally include determining parameters of a predefined model.

In Example 21, the subject matter of any one or any combination of Examples 16 to 20 may optionally include conducting a patient survey to determine the one or more patient dimensions of the neurostimulation.

In Example 22, the subject matter of any one or any combination of Examples 16 to 21 may optionally include determining the patient's response to the neurostimulation by programming an implantable stimulator to deliver the neurostimulation to the patient and measuring one or more parameters representing the patient's response.

In Example 23, the subject matter of determining the patient's response to the neurostimulation as found in Example 22 may optionally include producing one or more response curves each being a measured parameter of the measured one or more parameters plotted as a function of a stimulation parameter used by the implantable stimulator to control the delivery of the neurostimulation.

In Example 24, the subject matter of any one or any combination of Examples 16 to 23 may optionally further include delivering the neurostimulation to the patient according to the one or more optimal patterns of neurostimulation for a prolonged time period, receiving updated one or more patient dimensions during the prolong period according to a schedule, adjusting the personalized objective function using the updated one or more patient dimensions, adjusting the one or more optimal patterns of neurostimulation using the adjusted personalized objective function, and programming the stimulation device to deliver the neurostimulation according to the adjusted one or more optimal patterns of neurostimulation.

In Example 25, the subject matter of Examples 24 may optionally further include receiving updated patient's response to the neurostimulation, adjusting the personalized model to the updated patient's response to the neurostimulation, adjusting the one or more optimal patterns of neurostimulation using the adjusted personalized model, and programming the stimulation device to deliver the neurostimulation according to the adjusted one or more optimal patterns of neurostimulation.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an embodiment of a method for personalizing neurostimulation to a patient and delivering the personalized neurostimulation to the patient.

FIG. 10 illustrates an embodiment of a method for performing a patient assessment, such as an example of performing part of the method of FIG. 9.

FIG. 11 illustrates an embodiment of a method for analyzing and modeling, such as an example of performing part of the method of FIG. 9.

FIG. 12 illustrates an example of predicted and measured patient's responses for use in the modeling.

FIG. 13 illustrates an embodiment of a circuit for personalizing neurostimulation for a patient, such as an example of a circuit used to perform the method of FIG. 11.

FIG. 14 illustrates an embodiment of a method for optimizing one or more patterns of neurostimulation, such as an example of performing part of the method of FIG. 9.

FIG. 15 illustrates an embodiment of a circuit for optimizing personalized neurostimulation for the patient, such as an example of a circuit used to perform the method of FIG. 14.

FIG. 16 illustrates an embodiment of a method for delivering neurostimulation according to the optimized one or more patterns of neurostimulation, such as an example of performing part of the method of FIG. 9.

FIG. 17 illustrates an embodiment of a method for monitoring patient responses to the delivery of neurostimulation according to the optimized one or more patterns of neurostimulation, such as an example of performing part of the method of FIG. 9.

FIG. 18 illustrates an embodiment of a method for optimizing a pattern of neurostimulation for a patient.

FIG. 19 illustrates an embodiment of a circuit for optimizing a pattern of neurostimulation for a patient, such as an example of a circuit used to perform the method of FIG. 18.

FIG. 20 illustrates an example of results of performing the method of FIG. 18 using simulations.

FIG. 21 illustrates another example of results of performing the method of FIG. 18 using simulations.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a neurostimulation system that can be configured for delivering stimulation to a patient and for collecting information specific to the patient, personalizing a model to the patient, and optimizing neurostimulation for the patient using the collected information and the personalized model. In various embodiments, the neuromodulation system can include an implantable device configured to deliver neurostimulation (also referred to as neuromodulation) therapies, such as deep brain stimulation (DBS), spinal cord stimulation (SCS), peripheral nerve stimulation (PNS), and vagus nerve stimulation (VNS), and one or more external devices configured to program or adjust the implantable device for its operations and monitor the performance of the implantable device. While pain control using SCS is discussed as an example for illustrative purposes, the present subject matter can be applied to any indications that is to be treated using neurostimulation.

Pain is a patient-specific, idiosyncratic disease that may have multiple etiologies and may be driven by various neural circuits. Responses to SCS vary substantially from patient to patient, depending on specific physiological conditions, neural injuries, root causes, symptoms, etc. SCS waveforms that are effective for some circuits and/or pain states may not be effective on other circuits and/or pain states. Personalization of SCS waveforms for each patient has been difficult, for example due to the many variables involved. Optimization approaches have been undertaken to develop more effective and more consistent SCS waveforms. The resulting waveforms may perform optimally over one or more pain states but sub-optimally over other pain states. Approaches to optimize stimulation waveforms rapidly (e.g., in less than 6 hours) at reasonable costs using simulations with models of nervous system have been developed recently, making personalization of SCS waveforms for each patient technologically and economically feasible when suitable models can be determined.

The present subject matter provides for personalization of neurostimulation therapy, such as SCS therapy, for a patient by, for example, accounting for subjective inputs and response to pre-defined waveforms, determining a model-based optimization algorithm to develop a patient-specific neurostimulation pattern, and applying the resulting pattern in delivering neurostimulation to the patient. In various embodiments, the personalization can include the following processes: (1) determining the patient's condition indicator (e.g., the patient's pain state) from the patient's own responses to neurostimulation (e.g., SCS), (2) determining an objective function from information gathered from the patient, and (3) optimizing the patient's neurostimulation therapy (e.g., SCS therapy) and delivering the therapy in a fashion desired by the patient. In this document, the patient's “condition indicator” can be an indication of the patient's condition(s) that is measurable and used as an input into the model used in the optimization. For illustrative but not restrictive purposes, the patient's pain state is discussed in this document as an example of the patient's condition indicator, and SCS for pain control is discussed as an example of the neurostimulation. In various embodiments, the present subject matter can apply to neurostimulation for other indications and/or measures of the patient's conditions. In this document, the patient's “pain state” includes the state of the spinal cord network or physiology of the patient through which pain is represented by parameters that can be measured and used as an input into the model used in the optimization. The pain state can be inferred, for example when direct measurements from the spinal cord (beyond certain compound potentials) are difficult or impractical. The pain state can also imply pain etiology and/or disease condition. The “objective function” for the neurostimulation is a mathematical or quantitative expression of the patient's condition indicator (e.g., the patient's pain state) being a function of patient-specific information including patient input (e.g., answers to questions), signals sensed from the patient, and/or the like. An example of the objective function is the cost function in a machine learning algorithm. The objective function can be used to “grade” performance of a model or “incentivize” the model to develop towards a favorable outcome. Terms in the objective function can include elements (e.g. stimulation energy usage, patient sensation preference, etc.) beyond the spinal pain state. The pain state (and its response to the model-proposed intervention) can constitute one or multiple terms and/or weights of the objective function. The fashion desired by the patient can be determined using patient input, such as the patient's answers to a series of questions created for determining the patient's preferences.

In various embodiments, the present subject matter can be applied to control delivery of neurostimulation such that the patient's response to the delivery of the neurostimulation fits a known curve. Neurostimulation can to delivered to the patient according to a set of patterns of neurostimulation, one at a time. The patient's response curve (e.g., a sensed parameter being a measure of the patient's response to the delivered neurostimulation plotted against a stimulation parameter) can be determined. Patient-specific information can be collected to determine a personalized model for optimization of neurostimulation for the patient by selecting the model and/or tuning parameters of the model using the received patient-specific information. The personalized model can be optimized by tuning the model state such that a response curve derived using the model state closely matches the patient's response curve. An optimal pattern of neurostimulation can be determined using the optimized personalized model and the patient-specific information. The neurostimulation system can then deliver the neurostimulation according to the optimal pattern of neurostimulation.

In this document, an “optimal” pattern of neurostimulation includes a pattern of neurostimulation (defined by stimulation parameters) determined to maximize or minimize an objective function determined based on patient-specific information, such as determined to minimize a cost function determined based on patient-specific information. A model is “optimized” when the difference between a patient response predicted using the model and a patient response measured while delivering neurostimulation to the patient is minimized. An optimal pattern of neurostimulation can be a pattern selected from set of patterns of neurostimulation based on the differences determined for all the patterns in the set of patterns of neurostimulation.

In this document, unless noted otherwise, a “patient” includes a person receiving treatment delivered from, and/or being monitored and/or evaluated using, a neurostimulation system. A “user” includes a physician, other caregiver who examines, monitors, and/or treats the patient using the neurostimulation system, or other person who participates in the examination, monitoring, and/or treatment of the patient using the neurostimulation system (e.g., a technically trained representative, a field clinical engineer, a clinical researcher, or a field specialist from the manufacturer of the neurostimulation system).

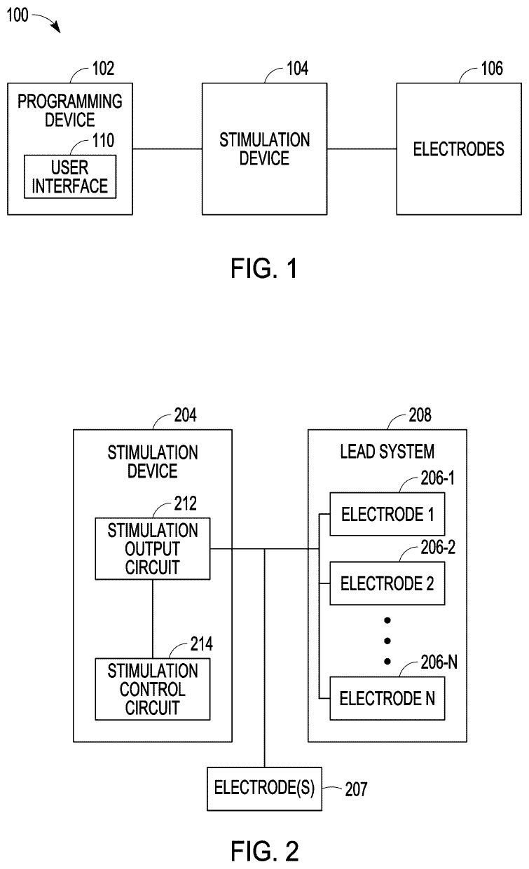

FIG. 1 illustrates an embodiment of a neurostimulation system 100. System 100 includes electrodes (also referred to as contacts) 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 110 can be configured for spinal cord stimulation (SCS) applications. Such SCS configuration includes various features that may simplify the task of the user in programming stimulation device 104 for delivering SCS to the patient, such as the features discussed in this document.

FIG. 2 illustrates an embodiment of a stimulation device 204 and a lead system 208, such as may be implemented in neurostimulation system 100. Stimulation device 204 represents an example of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 (also referred to as an electrode array in this document) distributed in the one or more leads. The plurality of electrodes 206 includes electrode 206-1, electrode 206-2, . . . electrode 206-N, each being a single electrically conductive contact providing for an electrical interface between stimulation output circuit 212 and tissue of the patient (and therefore also referred to as a contact in this document), where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

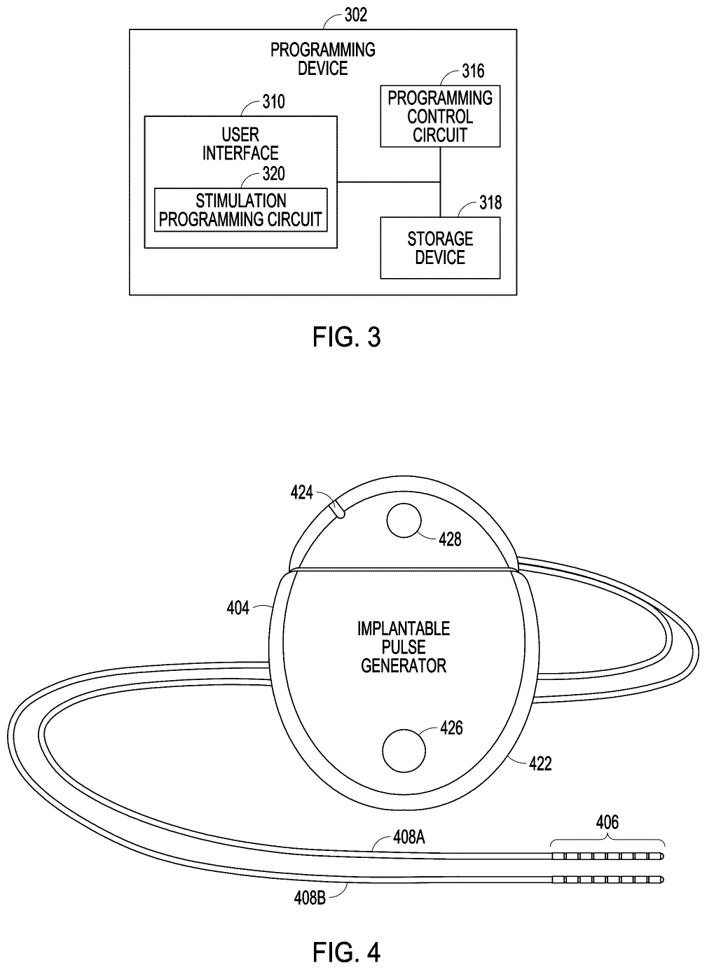

FIG. 3 illustrates an embodiment of a programming device 302, such as may be implemented in neurostimulation system 100. Programming device 302 represents an example of programming device 102 and includes a storage device 318, a programming control circuit 316, and a user interface 310. Programming control circuit 316 generates the plurality of stimulation parameters that controls the delivery of the neurostimulation pulses according to a specified neurostimulation program that can define, for example, stimulation waveform and electrode configuration. User interface 310 represents an example of user interface 110 and includes a stimulation programming circuit 320. Storage device 318 stores information used by programming control circuit 316 and stimulation programming circuit 320, such as information about a stimulation device that relates the neurostimulation program to the plurality of stimulation parameters. In various embodiments, stimulation programming circuit 320 can be configured to support one or more functions related to programming of stimulation devices, such as stimulation device 104 including its various embodiments as discussed in this document, including personalization of one or more patterns of neurostimulation for each patient.

In various embodiments, user interface 310 can allow for definition of a pattern of neurostimulation (e.g., a pattern of neurostimulation pulses) for delivery during a neurostimulation therapy session by creating and/or adjusting one or more stimulation waveforms using a graphical method. The definition can also include definition of one or more stimulation fields associated with the pattern of neurostimulation (e.g., one or more stimulation fields each associated with one or more pulses in the pattern of neurostimulation pulses). As used in this document, a “neurostimulation program” can include the pattern of neurostimulation including the one or more stimulation fields, or at least various aspects or parameters of the pattern of neurostimulation including the one or more stimulation fields. In various embodiments, user interface 310 includes a GUI that allows the user to define the pattern of neurostimulation and perform other functions using graphical methods. In this document, “neurostimulation programming” can include the definition of the one or more stimulation waveforms, including the definition of one or more stimulation fields.

In various embodiments, circuits of neurostimulation system 100, including its various embodiments discussed in this document, may be implemented using a combination of hardware and software. For example, the circuit of user interface 110, stimulation control circuit 214, programming control circuit 316, and stimulation programming circuit 320, including their various embodiments discussed in this document, can be implemented using an application-specific circuit constructed to perform one or more particular functions and/or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrodes (also referred to as contacts) 406 for contacting a tissue site targeted for electrical neurostimulation. As illustrated in FIG. 4, leads 408A and 408B each include 8 electrodes 406 at the distal end. The number and arrangement of leads 408A and 408B and electrodes 406 as shown in FIG. 4 are only an example, and other numbers and arrangements are possible. In various embodiments, the electrodes are ring electrodes. In various embodiments applying DBS or SCS, the implantable leads and electrodes may be configured by shape and size to provide electrical neurostimulation energy to a neuronal target included in the patient's brain or configured to provide electrical neurostimulation energy to target nerve cells in the patient's spinal cord.

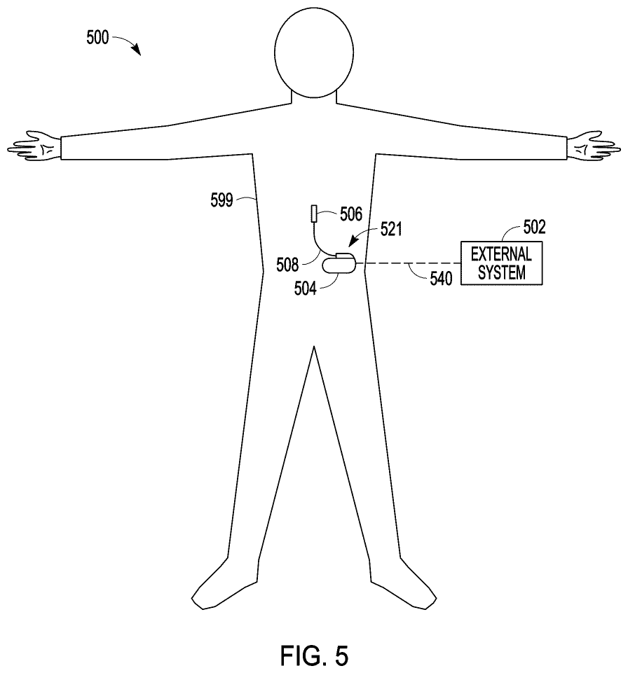

FIG. 5 illustrates an implantable neurostimulation system 500 and portions of an environment in which system 500 may be used. System 500 includes an implantable system 521, an external system 502, and a telemetry link 540 providing for wireless communication between implantable system 521 and external system 502. Implantable system 521 is illustrated in FIG. 5 as being implanted in the patient's body 599.

Implantable system 521 includes an implantable stimulator (also referred to as an implantable pulse generator, or IPG) 504, a lead system 508, and electrodes (also referred to as contacts) 506, which represent an example of stimulation device 204, lead system 208, and electrodes 206, respectively. External system 502 represents an example of programming device 302. In various embodiments, external system 502 includes one or more external (non-implantable) devices each allowing the user and/or the patient to communicate with implantable system 521. In some embodiments, external 502 includes a programming device intended for the user to initialize and adjust settings for implantable stimulator 504 and a remote control device intended for use by the patient. For example, the remote control device may allow the patient to turn implantable stimulator 504 on and off and/or adjust certain patient-programmable parameters of the plurality of stimulation parameters.

The sizes and shapes of the elements of implantable system 521 and their location in body 599 are illustrated by way of example and not by way of restriction. An implantable system is discussed as a specific application of the programming according to various embodiments of the present subject matter. In various embodiments, the present subject matter may be applied in programming any type of stimulation device that uses electrical pulses as stimuli, regarding less of stimulation targets in the patient's body and whether the stimulation device is implantable.

Returning to FIG. 4, the IPG 404 can include a hermetically-sealed IPG case 422 to house the electronic circuitry of IPG 404. IPG 404 can include an electrode 426 formed on IPG case 422. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode. Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

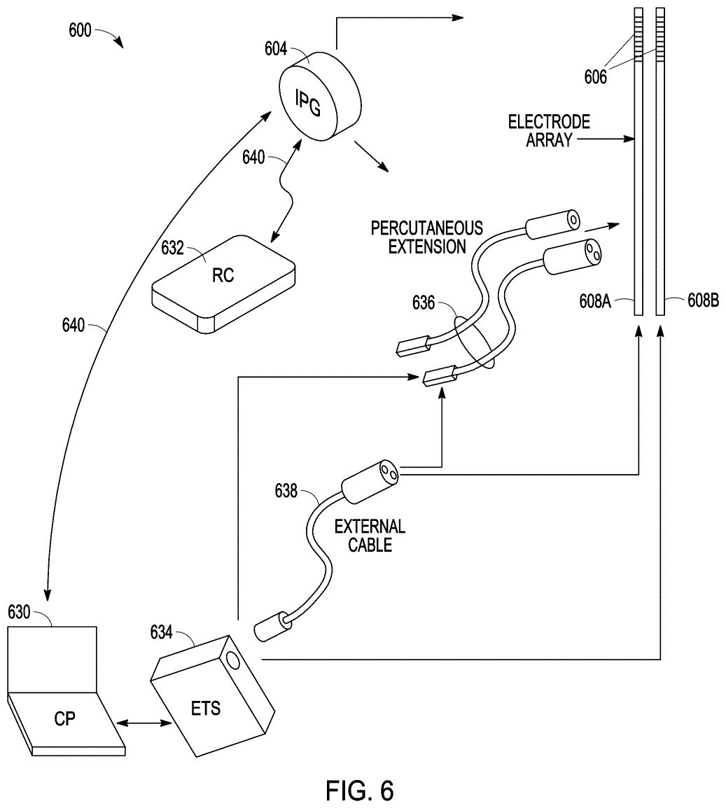

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (ETS, also referred to as external trial modulator, ETM) 634. IPG 604 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an example of system 100, with IPG 604 representing an embodiment of stimulation device 104, electrodes (also referred to as contacts) 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2π, where λ is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604.

RC 632 also communicates with IPG 604 using a wireless link 640. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630. CP 630 may communicate with the RC 632 using a wired or wireless communications link. In some embodiments, CP 630 can program RC 632 when remotely located from RC 632. In various embodiments, RC 632 can be a dedicated device or a general-purpose device configured to perform the functions of RC 632, such as a smartphone, a tablet computer, or other smart/mobile device.

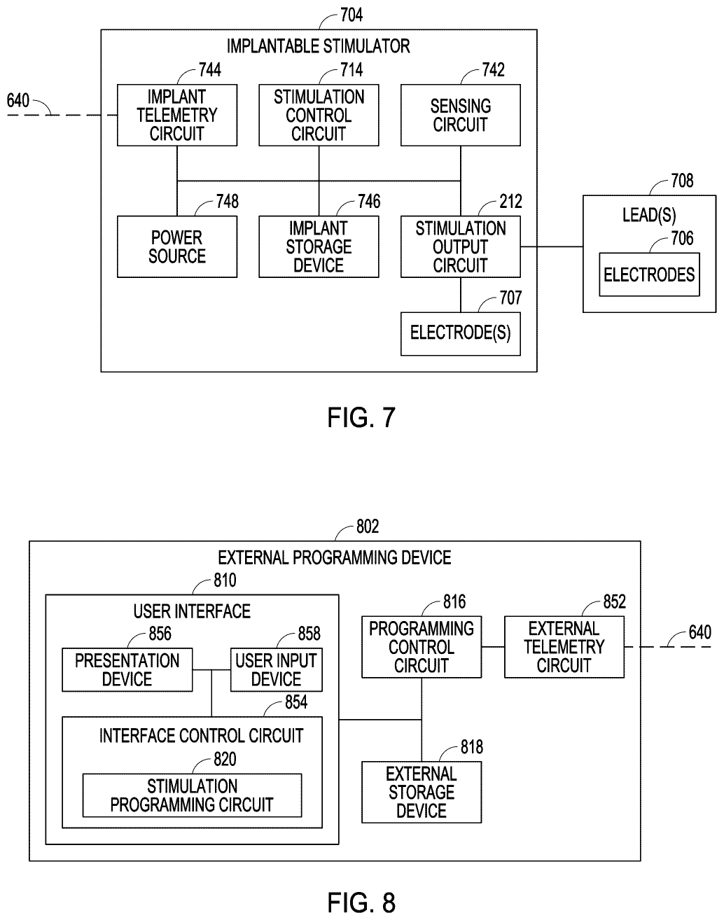

FIG. 7 illustrates an embodiment of implantable stimulator 704 and one or more leads 708 of an implantable neurostimulation system, such as implantable system 600. Implantable stimulator 704 represents an example of stimulation device 104 or 204 and may be implemented, for example, as IPG 604. Lead(s) 708 represents an example of lead system 208 and may be implemented, for example, as implantable leads 608A and 608B. Lead(s) 708 includes electrodes (also referred to as contacts) 706, which represents an example of electrodes 106 or 206 and may be implemented as electrodes 606.

Implantable stimulator 704 may include a sensing circuit 742 that provides the stimulator with a sensing capability, stimulation output circuit 212, a stimulation control circuit 714, an implant storage device 746, an implant telemetry circuit 744, a power source 748, and one or more electrodes 707. Sensing circuit 742 can one or more physiological signals for purposes of patient monitoring and/or feedback control of the neurostimulation. In various embodiments, sensing circuit 742 can sense one or more ESG signals using electrodes placed over or under the dura of the spinal cord, such as electrodes 706 (which can include epidural and/or intradural electrodes). In addition to one or more ESG signals, examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707 and delivers each of the neurostimulation pulses through a set of electrodes selected from electrodes 706 and electrode(s) 707. Stimulation control circuit 714 represents an example of stimulation control circuit 214 and controls the delivery of the neurostimulation using the plurality of stimulation parameters specifying the pattern of neurostimulation. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 can store one or more neurostimulation programs and values of the plurality of stimulation parameters for each of the one or more neurostimulation programs. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 (e.g., by installing a new application in a smart device such as a smartphone) and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640 and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing circuit 742.

In various embodiments, sensing circuit 742 (if included), stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an example of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). This can allow for patient mobility during programming and assessment when needed. For example, wireless communication link 640 can include at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a DBS or SCS therapy session, as well as various parameters and building blocks for defining one or more waveforms. The one or more stimulation waveforms may each be associated with one or more stimulation fields and represent a pattern of neurostimulation to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time (and allows the user to do so) to shape the stimulation like a piece of clay (pushing/pulling one spot at a time).

Programming control circuit 816 represents an example of programming control circuit 316 and generates the plurality of stimulation parameters, which is to be transmitted to implantable stimulator 704, based on a specified neurostimulation program (e.g., the pattern of neurostimulation as represented by one or more stimulation waveforms and one or more stimulation fields, or at least certain aspects of the pattern). The neurostimulation program may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an example of user interface 310 and allows the user to define the pattern of neurostimulation and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes a stimulation control circuit 820.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. Programming control circuit 816 dynamically updates values of the plurality of stimulation parameters in response to changes in the set of one or more stimulation waveforms and transmits the plurality of stimulation parameters with the updated values to implantable stimulator 704.

Stimulation control circuit 820 represents an example of stimulation programming circuit 320 and can be configured to provide for closed-loop control of steering of stimulation field according to the present subject matter. In various embodiments, User interface 810 can be configured to allow the user and/or the patient to drive the steering, with stimulation control circuit 820 adjusting the stimulation parameters for substantially maintaining a level of paresthesia while the stimulation field is being moved.

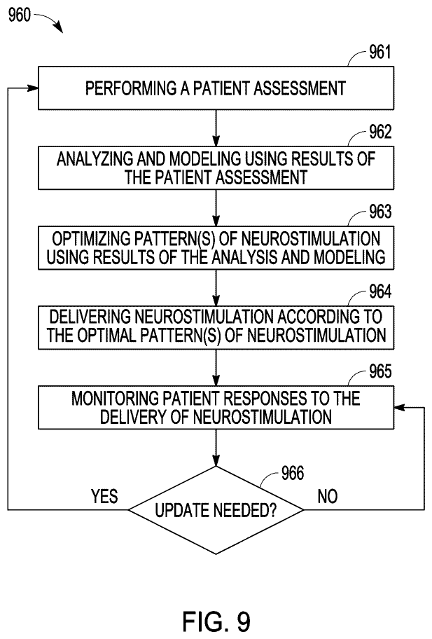

FIG. 9 illustrates an embodiment of a method 960 for personalizing neurostimulation to a patient and delivering the personalized neurostimulation to the patient. Method 960 can be performed using system 100, including its various embodiments (e.g., system 500 or 600) and various embodiments of its components. For example, method 960 can be performed using implantable stimulator 704, such as implemented as IPG 404, 504, or 604, and external programming device 802, such as implemented in CP 630, RC 632 (e.g., implemented as a dedicated device or in a smart phone), and/or one or more other devices (e.g., chosen from mobile devices, laptop computers, tablet computers, or other computerized devices).

At 961, a patient assessment is performed. The patient assessment can include a patient survey and a calibration. The patient survey can be conducted to determine one or more patient dimensions of the neurostimulation. The calibration can be performed to determine the patient's response to the neurostimulation by delivering the neurostimulation from a stimulation device to the patient and measuring one or more parameters representing the patient's response. The patient's response can include, for example, one or more response curves each being a measured parameter representing the patient's response plotted as a function of a stimulation parameter (or a set of stimulation parameters, patterns, waveforms, or other aspects of the neurostimulation suitable for evaluating the patient's response) used to control the delivery of the neurostimulation. In one example, external programming device 802 is used for performing the patient assessment, with stimulation programming circuit 820 configured (e.g., programmed) for controlling the patient survey and the calibration. The patient survey can be conducted using user interface 810. The calibration can be performed by using external programming device 802 to program implantable stimulator 704 for delivering the neurostimulation and to measure the one or more parameters representing the patient's response to the delivery of the neurostimulation.

At 962, results of the patient assessment are analyzed, and a model is determined. Patient-specific information including the one or more patient dimensions of the neurostimulation and calibration information including the patient response to the neurostimulation are received and analyzed. A personalized objective function for the neurostimulation is determined for the patient using the one or more patient dimensions. An example of the personalized objective function is a personalized cost function being the patient's condition indicator (e.g., a quantitative measure of the patient's condition such as the pain state and/or one or more other indicators of conditions related to pain or pain control) expressed as a function of the one or more patient dimensions. A personalized model having a model state personalized to the patient's condition indicator, including the patient's response to the neurostimulation, is determined for the patient. The personalized model (e.g., a computational model) allows for prediction of physiological responses (e.g., neural responses) to a pattern of neurostimulation using simulation. A “model state” is an instance of the computational model parameterized specifically for a patient or a condition (e.g., a set of one or more neurological characteristics) of the patient. In one embodiment, the personalized model is optimized by minimizing the difference between the patient's response to the neurostimulation as predicted by simulation using the personalized model and the patient's response as detected during actual delivery of the neurostimulation is minimized. This minimization of the different can be performed by determining or selecting a model state for matching the its response to the simulated neurostimulation to the patient's response to actual delivery of the neurostimulation. In one example, the analysis and modeling are performed by external programming device 802, such as using stimulation programming circuit 820 that is implemented in CP 630 and/or a device communicatively coupled to CP 630, depending on design considerations such as computing power and/or user expertise required for the front end devices such as CP 630.

At 963, one or more patterns of neurostimulation are optimized using results of the analysis and modeling. This optimization can include determining one or more optimal patterns of neurostimulation using the personalized objective function and the personalized model. An “optimal” pattern of neurostimulation is a pattern of neurostimulation (defined by stimulation parameters) determined to maximize or minimize the personalized objective function, depending on the nature of the objective function (e.g., to minimize when objective function is a cost function). The optimization can include adjusting one or more existing patterns of neurostimulation or selecting the one or more optimal patterns of neurostimulation from multiple existing patterns of neurostimulation. In various embodiments, the one or more optimal patterns of neurostimulation can represent a therapy approach optimized for the patient. Each optimal pattern of neurostimulation can include one or more optimal stimulation parameters defining stimulation waveform, field, and/or timing of delivery. The optimization can be performed, for example, using a machine learning algorithm. In one example, the optimization can be performed by external programming device 802, such as using stimulation programming circuit 820 that is implemented in CP 630 and/or a device communicatively coupled to CP 630, depending on design considerations such as computing power and/or user expertise required for the front end devices such as CP 630. In one example, the optimization is performed for a patient in a clinic, using a clinician's programmer in the clinic (e.g., CP 630) and a remote device (e.g., a computer in a remote site) that can be communicatively coupled to the clinician's programmer via a telecommunication network. The remote device can be used to perform the analysis and modeling to determine the personalized objective function and the personalized model in the remote site. The clinician's programmer or the remote device can be used to perform the optimization to determine the one or more optimal patterns of neurostimulation, depending on design considerations (e.g., computing power of the clinician's programmer and/or expertise required for the clinic).

At 964, neurostimulation is delivered to the patient according to the optimized one or more patterns of neurostimulation. A stimulation device (e.g., the stimulation device used in the calibration) can be programmed to deliver the neurostimulation to the patient and to control the delivery according to the one or more optimal patterns of neurostimulation. In one example, external programming device 802 (e.g., portions implemented in CP 630) is used to program implantable stimulator 704 (e.g., implemented as IPG 604) to deliver the neurostimulation to the patient according to the optimized one or more patterns of neurostimulation.

At 965, the patient's response to the delivery of neurostimulation is monitored. The neurostimulation can be delivered to the patient for a prolonged period using the one or more optimal pattern of neurostimulation. During the prolonged period, the patient is evaluated according to a schedule (e.g., periodically) and/or when a need is indicated (e.g., the therapy stops being effective). The evaluation can be designed for detecting indications for need to update the one or more optimal patterns of neurostimulation. If the need to update is not indicated at 966, the patient's response to the delivery of neurostimulation is continued to be monitored at 965. If the need to update is indicated at 966, the process including steps 961-965 can be repeated.

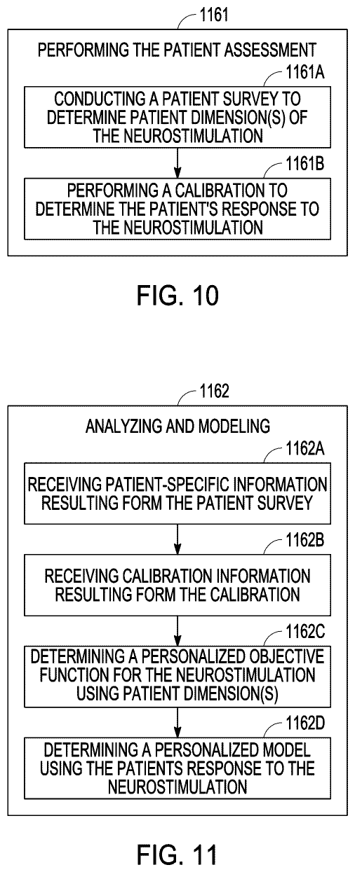

In various embodiment, method 960 can be performed throughout a neurostimulation therapy for the patient such that the therapy remains personalized and optimized for the patient. Various steps of method 960 are further discussed below with reference to FIGS. 10-17.

FIG. 10 illustrates an embodiment of a method 1061 for performing a patient assessment, such as an example of performing step 961 of method 960. When the patient is initially enrolled, or upon a patient or clinician-requested update, an assessment can be performed, e.g., in a clinic into which the patient is brought. The patient is subjected to the patient survey and the calibration.

At 1061A, a patient survey is conducted to determine one or more patient dimensions of neurostimulation that matter more (or less) for formulation of an objective function and also to parameterize a model according to properties of the patient's pain beyond one or more pain scores such as numerical rating scale (NRS, e.g., 1-10) or visual analog scale (VAS). The patient survey can include questionnaires and/or one or more other forms of questioning or interviewing using established metrics, such as the Guided Sensation Questionnaires. The patient survey can be conducted through a digital health application, e.g., via existing infrastructure. The patient survey can include free text and/or transcribed voice that is used to infer one or more variables of interest. Examples of the one or more patient dimensions of neurostimulation can include any one or any combination of the following:
pain score metrics such as NRS (e.g., 1-10 for lowest to highest pain intensity, VAS (e.g., a pain intensity sliding bar), numerically-graded subsets (e.g., rating certain pain-associated sensations), and the like;
affective/emotional metrics such as Oswestry Disability Index, Beck Depression Inventory (BDI), 36-Item Short Form Survey (SF-36), and the like;
measures of function such as Activities of Daily Living (ADLs), return-to-work (RTW), fitness goals, activity tracking, measures of sleep quality, and the like;
measures of community/social engagement, both with respect to social activity and/or managing social perceptions of receiving stimulation;
patient tolerability of SCS independent to emotional/affective dimension of pain, possibly a composite metric;
measures related to stimulation: paresthesia vs. no paresthesia, desire to overlap paresthesia with pain, desired recharging frequency, desired sensations if paresthesia, desired dose (e.g., program scheduling or cycling, independent of recharge); and
measures or descriptors of other symptoms affected by or related to the neurostimulation, such as autonomic modulation (e.g., bladder, bowel, and/or sexual side effects and changes in heart rate and/or blood pressure), motor function affected by the neurostimulation, and/or potential interference (beneficial or adverse) by the neurostimulation with other pre-existing conditions that are not treated by the neurostimulation (e.g., effects of SCS on conditions other than pain).
   Multiple patient dimensions can be paired with an “importance” metric or weighting factors each representing relative importance of a dimension of the multiple patient dimensions to the patient.

At 1061B, a calibration is performed to determine the patient's response to the neurostimulation. The calibration can start with determining a set of patterns of neurostimulation for inferring one or more response curves and/or one or more response maps for the patient. The one or more response curves and/or one or more response maps each relate a measure of response to a pattern of neurostimulation, which can be characterized by a stimulation waveform or a variable stimulation parameter of the stimulation waveform (e.g., pulse frequency). The calibration can be waived if a model-etiology relationship is already known for the patient. The neurostimulation is delivered to the patient according to the set of patterns of neurostimulation, one at a time. The patient's response is evaluated, for example by rating rapid-onset pain relief (or not, e.g., by asking the patient to rate) from each pattern in the set of patterns of neurostimulation. A response curve can be generated by plotting a measure of the patient's response (e.g., the rating of pain relief) against a stimulation parameter. In various embodiments, the set of patterns of neurostimulation can include non-regular, modulated, and/or other non-tonic waveforms. Waveforms used in the set of patterns of neurostimulation can be pre-defined and designed for network state calibration (i.e., for characterizing relevant portions of the patient's neuronal network) rather than efficacy. The patient's responses to various waveforms can then be used to personalize a model to the patient.

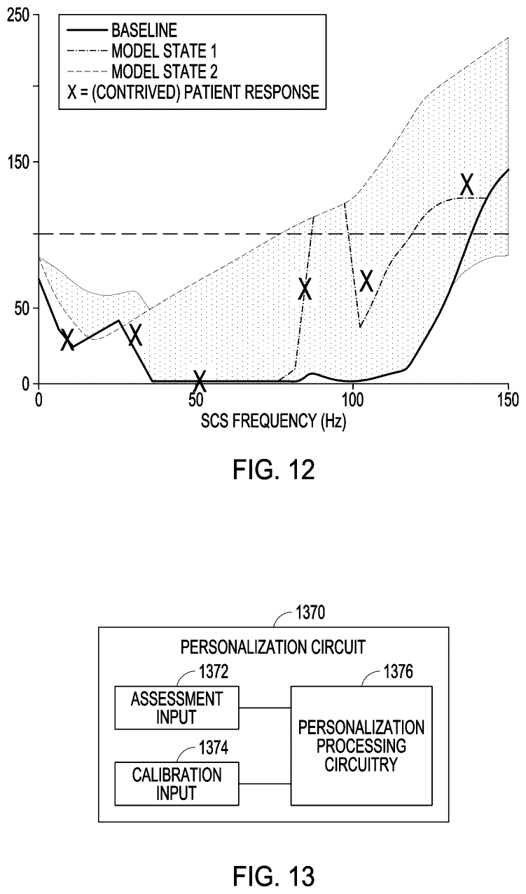

FIG. 11 illustrates an embodiment of a method 1162 for analyzing and modeling, such as an example of performing step 962 of method 960. Method 1162 can be performed to determine a personalized objective function and a personalized model for the patient, for example using results from performing method 1061. FIG. 12 illustrates an example of predicted and measured patient's responses for use in the modeling. In the illustrated example, a measure of the patient's response to SCS is plotted against the pulse frequency used in the SCS. The vertical axis is the firing of a model pain neuron, expressed as a percentage of firing when SCS is off (“baseline”). The model neuron's firing can be taken as a proxy for pain (e.g., VAS; however any or all of the metrics discussed can be used if plottable). The illustrated response curves are each derived from a model with a model state using simulations. The patient's response obtained from the calibration is also plotted against the pulse frequency. In the illustrated example, the patient's response is closest to the response curve derived using the model with model state 1.

FIG. 13 illustrates an embodiment of a personalization circuit 1370 that can be configured (e.g., programmed) to perform method 1162. Personalization circuit 1370 can include an assessment input 1372, a calibration input 1374, and personalization processing circuitry 1376. In various embodiments, personalization circuit 1370 can be implemented in external programming device 802, such as using stimulation programming circuit 820 that is implemented in CP 630 and/or a device communicatively coupled to CP 630, depending on design considerations.

Assessment input 1372 can receive patient-specific information resulting from a patient survey, such as a performance of step 1061A of method 1061. The patient-specific information includes the one or more patient dimensions of the neurostimulation, such as discussed for step 1061A. Calibration input 1374 can receive calibration information resulting from a patient calibration, such as a performance of step 1061B of method 1061. The calibration information includes the patient's response to the neurostimulation, such as discussed for step 1061B. Personalization processing circuitry 1376 can determine a personalized objective function using the received one or more patient dimensions and determine a personalized model (e.g., a personalized computational model) having a model state personalized to the patient's condition indicator including the patient's response to the neurostimulation.

Referring back to FIG. 11, at 1162A, the patient-specific information resulting from the patient survey is received. The patient-specific information includes at least the one or more patient dimensions of the neurostimulation. At 1162B, the calibration information resulting from the calibration is received. The calibration information includes at least the patient's response to the neurostimulation (e.g., one or more patient response curves).

At 1162C, the personalized objective function is determined using the results of the patient survey including the one or more patient dimensions of the neurostimulation. In one embodiment, the personalized objective function is selected from multiple predefined objective functions using the results of the patient survey. In various embodiments, the personalized objective function is a cost function (CF) used for optimization. In one embodiment, the CF is determined using a common template, for example:
CF=Σₖ₌₁^{N} Aₖ,
   where the relative weight/contribution of the kth factor A is previously described as

$${A}_{k} = ( {\sum }_{j = 1}^{M} {B}_{j} ) \cdot w \cdot metric ,$$

where Bⱼ is the score from the patient's answer to question j among M questions, and w is the baseline or default weighting assigned to a given “metric”, or potentially the weighting established by the answer to the “importance” question. That weighting can be a discrete value (e.g. 0, 1, 2, or 3) or a slider. It is noted that the CF need not be a simple summation, and weights need not be linear. Weights could be squared, exponential, etc . . . , and the “combination” could be a product, a division operation, or another method of scaling. In another embodiment, the CF is selected from multiple pre-defined CFs using the results of the patient survey. Several pre-defined cost functions can be made explicitly available, and questions in the patient survey can be routed through a machine learning algorithm, a decision tree, or a look-up table to determine which CF to select. In some examples, multiple CF blocks can be chosen, and an absolute or relative weighting may be assigned to each block based on answers to the questions in the patient survey. In some embodiments, the CF can be defined, or selected from multiple pre-defined CFs, using information other than answers to questions of the patient survey. For example, the user can explicitly define the objective function, or the CF can be assembled based on one more sensed or detected biomarkers (e.g. EEG, ESG, heart rate, HRV, activity levels, and the like, such as sensed during the calibration) instead of or in addition to using the answers to the questions of the patient survey.

At 1162D, the personalized model is determined using the results of the calibration including the patient's response to the neurostimulation. A model, such as a computational model, can be derived clinically and/or based on published information (e.g., literature and reports). This model is then personalized using the calibration information to best match the relevant portions of the patient's neuronal network. The personalized model is the model in a model state personalized for the patient (i.e., an instance of the computational model with a given set of model parameters individually determined for the patient or the patient's condition). The model state substantiates the model for the patient. The model states of a model are instances of the model with different sets of model parameters. In one embodiment, a new set of the model parameters are determined, resulting a new model state being the model state of the personalized model. In another embodiment, a model state is selected from pre-defined model states (e.g. in FIG. 12, model state 1 can be selected) to be the model state of the personalized model. This can include comparing the patient's response curve(s) to stored response curves derived from previously characterized model states and selecting the model state by matching the curves. In various embodiments, the personalization model can be optimized to minimize a difference between the responses derived from its model state and the patient responses measured during the calibration. The optimization can include a process of reconciling patient-specific parameters with the selected model. The model can be a neuronal network model. In various embodiments, the model can extend to include, for example, axon simulations with coefficient of variation, a clinically/empirically derived model, a machine learning model, and/or another approach beyond biophysical simulation. The patient-specific parameters are each related to one or more parameters of the set of model parameters. Examples of the patient-specific parameters include pain etiology, pain duration, pain location, emotional/affective (e.g., supraspinal input) responses to SCS waveforms, and the like. An example of the model is discussed in Zhang et al., “Modeling effects of spinal cord stimulation on wide-dynamic range dorsal horn neurons: influence of stimulation frequency and GABAergic inhibition”, Journal of neurophysiology, 112 (3), pp. 552-567 (2014) and Gilbert et al., “Evaluating optimized temporal patterns of spinal cord stimulation (SCS)”, Brain Stimulation, 15 (5), pp. 1051-1062 (2022), which are incorporated herein by reference.

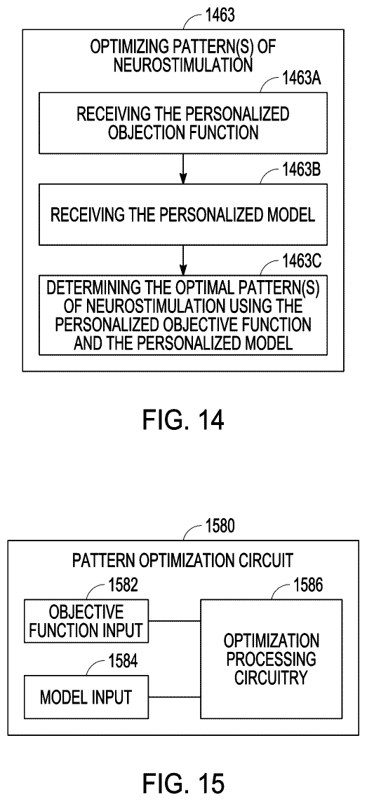

FIG. 14 illustrates an embodiment of a method 1463 for optimizing one or more patterns of neurostimulation, such as an example of performing step 963 of method 960. Method 1463 can be performed to determine one or more optimal patterns of neurostimulation for the patient, for example using results from performing method 1162.

FIG. 15 illustrates an embodiment of a pattern optimization circuit 1580 that can be configured (e.g., programmed) to perform method 1463. Pattern optimization circuit 1580 can include an objective function input 1582, a model input 1584, and optimization processing circuitry 1586. In various embodiments, pattern optimization circuit 1580 can be implemented in external programming device 802, such as using stimulation programming circuit 820 that is implemented in CP 630 and/or a device communicatively coupled to CP 630, depending on design considerations.

Objective function input 1582 can receive the personalized objective function, such as resulting from performing step 1162C of method 1162. Model input 1584 can receive the personalized model, such as resulting from performing step 1162D of method 1162. Optimization processing circuitry 1586 can determine the one or more optimal patterns of neurostimulation using the personalized objective function and the personalized model. The one or more optimal patterns of neurostimulation can be used to program a stimulation device for controlling delivery of the neurostimulation to the patient.

Referring back to FIG. 14, at 1463A, the personalized objective function is received. At 1463B, the personalized model is received. At 1463C, the one or more optimal patterns of neurostimulation are determined using the personalized objective function and the personalized model. The determination of the one or more optimal patterns of neurostimulation can be part of optimization of a therapy approach for the patient. In various embodiments, an optimization is performed using an optimal personalized model (i.e., the personalized model whose responses to stimulation parameters best match the patient's respective responses). The optimization can be performed using an appropriate machine learning algorithm (e.g. particle swarm, GA, random forest, or the like). Output of the optimization includes the one or more optimal patterns of neurostimulation or aspects of the one or more optimal stimulation patterns, such as a “best” waveform, a range of parameters (akin to Neural Dosing ranges), the top few “organisms” in a program schedule, and the like. In one embodiment, the one or more optimal patterns of neurostimulation or the aspects of the one or more optimal patterns of neurostimulation are evaluated against existing patters of neurostimulation or respective aspects of the existing patterns of neurostimulation using the personalized model. The one or more optimal patterns of neurostimulation are determined using the outcome of the evaluation (e.g., selected from the existing patters of neurostimulation or determined using aspects selected from the respective aspects of the existing patterns of neurostimulation.

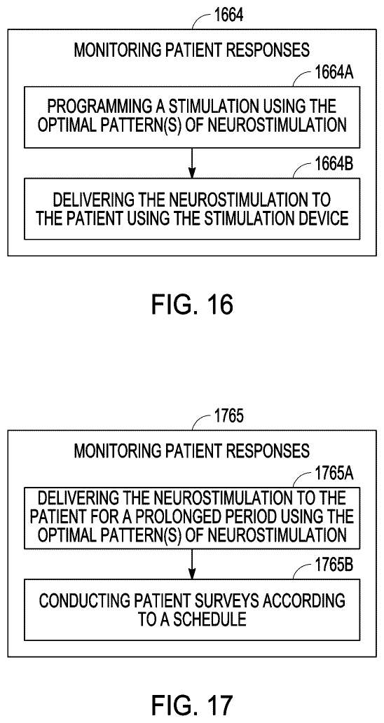

FIG. 16 illustrates an embodiment of a method 1664 for delivering neurostimulation according to the optimized one or more patterns of neurostimulation, such as an example of performing step 964 method 960. Method 1664 can be performed to deliver the neurostimulation and to control the delivery of the neurostimulation using, for example, results from performing method 1463.

At 1664A, a stimulation device is programmed using the one or more optimal patterns of neurostimulation. An example of the stimulation device includes implantable stimulator 704, such as implemented as IPG 404, 504, or 604, which can be programmed using, for example external programming device 802, such as implemented in CP 630, RC 632, and/or one or more other devices. In various embodiments, a neurostimulation program is determined for the patient using the one or more optimal patterns of neurostimulation. For example, the neurostimulation program can include delivering neurostimulation according to one optimal pattern of neurostimulation at a time, with a schedule defining when each optimal pattern of neurostimulation is applied. In various embodiments, the neurostimulation program can be determined for the patient using the one or more optimal patterns of neurostimulation and patient-specific information such as pain etiology, answers to certain questions in the patient survey, and the patient's responses to SCS patterns. Decision tree, look-up table, over-riding questions, and the like can be used in various processes of the determination. Examples of patient-specific information that can be used in determining the neurostimulation program include:
dorsal horn vs. dorsal column etiology that may dictate stimulation field (e.g., electrode geometry);
patient preference for amplitude (perceptive or sub-perceptive) and/or recharge frequency that may dictate or govern intensity and/or contribute to cost function;
answers related to sensation quality and/or emotional-affective dimensions of pain that may cause a neurostimulation algorithm to emphasize modulation and/or dynamic stimulation over static stimulation;
patient preference for waveforms and patient's activities or postures that may toggle periodic sensing as “surveillance” and/or incorporation of accelerometer and wearable data; and
patient-provided daily schedule that may affect timing of delivery of neurostimulation.

At 1664B, the neurostimulation is delivered to the patient using the stimulation device. The delivery is controlled according to the neurostimulation program, including the one or more optimal patterns of neurostimulation.

FIG. 17 illustrates an embodiment of a method 1765 for monitoring patient responses to the delivery of neurostimulation according to the one or more optimal patterns of neurostimulation, such as an example of performing step 965 of method 960. Method 1765 can be performed to monitor the patient's response to a prolonged performance of method 1664.

At 1765A, the neurostimulation is delivered to the patient for a prolonged time using the one or more optimal patterns of neurostimulation (e.g., a period of time sufficient long for observing and understanding the effects of the one or more optimal patterns of neurostimulation in the patient). At 1765B, patient surveys are conducted according to a schedule (e.g., periodically) and/or as needed (e.g., in response to a patient request). These surveys can be designed to determine whether the one or more optimal patterns of neurostimulation need to be updated (e.g., re-optimized) and can be simpler than the survey conducted at 961 or 1061A. In various embodiment, in addition to or in place of these surveys, internally sensed biomarkers (e.g., detection of gross lead migration or impedance change) can be used to determine whether the one or more optimal patterns of neurostimulation need to be updated. In response to a determination that the one or more optimal patterns of neurostimulation need to be updated, steps 961-965 of method 960 can be repeated to re-optimize the one or more optimal patterns of neurostimulation.

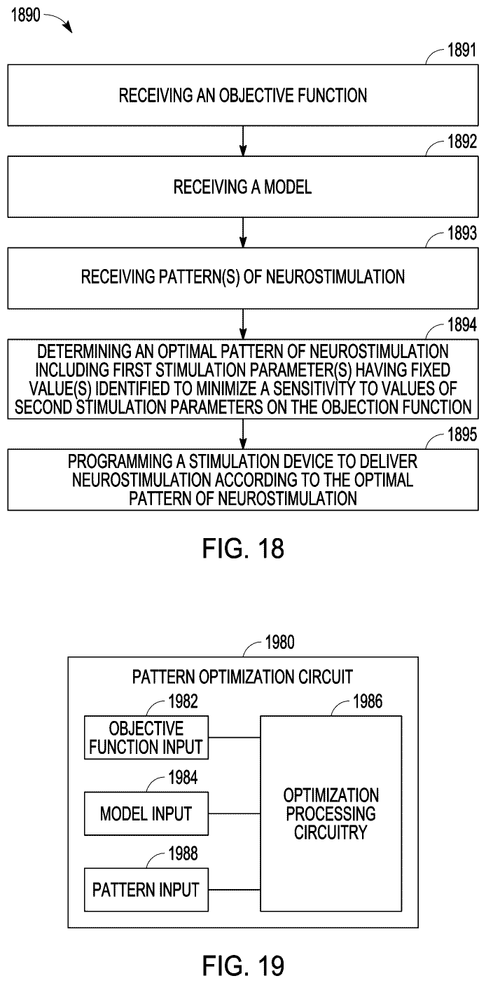

FIGS. 18-21 show an application of method 960 (including various embodiments of its steps as discussed above with reference to FIGS. 10-17) in programming neurostimulation for a patient. In SCS, proper stimulation programming is very important for providing the patient with significant therapeutic benefit without significant adverse side effects. Improper stimulation programming can result in effects that are harmful to the patient. The stimulation programming can often be difficult because, for example, the user must determine values of one or more stimulation parameters for overlapping paresthesia with area of pain while simultaneously controlling a vast array of other stimulation parameters. Such stimulation parameters (e.g., pulse amplitude, pulse width, and pulse frequency) may each be modulated, for example by a low frequency sinusoidal wave, to result in a dynamic stimulation pattern. A way for facilitating the stimulation programming by reducing the burden on the user in simultaneously determining values of multiple stimulation parameters is needed.

As an application of the present subject matter including the model optimization as discussed above, an optimized model can be used to test large ranges of various stimulation parameters with simulations, thereby substantially reducing the amount of parameter values that need to be tested on the patient. In one example, the model is used to traverse the parameter space and find a family of optimal dynamic patterns of stimulation that can significantly reduce pain for a large range of stimulation amplitudes. Patient-specific information can be collected (e.g., at a clinic) and used to determine a personalized model. The personalized model can be used to generate patterns of neurostimulation (e.g., at the clinic or a remote analysis center) specifically for the patient. Neurostimulation is delivered to the patient according to these patterns of neurostimulation (e.g., one at a time) for determining a therapy program (e.g., one or more stimulation parameter values) for the patient.

In this document, a “tonic stimulation” or “tonic pattern of neurostimulation” includes a pattern of neurostimulation with constant stimulation parameters (e.g., pulse amplitude, pulse width, and pulse frequency each given a constant value). A “dynamic stimulation” or “dynamic pattern of neurostimulation”: a pattern of neurostimulation with one or more stimulation parameters each being modulated by a time-varying signal (e.g., pulse amplitude, pulse width, and/or pulse frequency each expressed as a function of time).

FIG. 18 illustrates an embodiment of a method 1890 for optimizing a pattern of neurostimulation for a patient. The optimization can include fixing values for certain stimulation parameters to reduce the need to evaluate values of these stimulation parameters by actually delivering the neurostimulation while sweeping values of various parameters during a programming session with the patient. Simulations using the personalized objective function and the personalized model, as discussed above, can be used to identify such stimulation parameters and their values. For example, it was found that when several stimulation parameters are set to certain values, efficacy of pain control can be maintained for a wide range of pulse amplitude. Thus, method 1890 can be performed to improve efficiency of programming the neurostimulation for the patient by reducing the number of stimulation parameter to be evaluated during the programming session. Method 1890 can be performed, for example, as part of method 1463, in which the determination of the one or more optimal patterns of neurostimulation includes identifying stimulation parameters whose values can be fixed and determining such values.

At 1891, an objective function is received. The objective function can be the personalized objective function for the patient as discussed above. At 1892, a model (e.g., a computational model) is received. The model can be the personalized model for the patient as discussed above. The objective function and the model can be determined, for example, by performing methods 1061 and 1062. At 1893, one or more patterns of neurostimulation are received. The one or more patterns of neurostimulation each include multiple stimulation parameters whose values can be optimized using a combination of computer simulations (including performing method 1890) and patient evaluations (including delivering the neurostimulation to the patient with values of stimulation parameters to be tested). The one or more patterns of neurostimulation can each be a pattern of neurostimulation pulses. In various embodiments, the one or more patterns of neurostimulation pulses can include one or more dynamic patterns of neurostimulation each including at least one stimulation parameter that is modulated by a modulating waveform. A pattern of neurostimulation is “optimized” or “optimal” for the purpose of performing method 1890 when at least one stimulation parameter is identified using a computer simulation for having a fixed value that can be used in a patient evaluation to eliminate the need for determining a value for that stimulation parameter during the patient evaluation.

At 1894, an optimal pattern of neurostimulation is determined using a computer simulation with the objective function, the model, and the one or more patterns of neurostimulation. The optimal pattern of neurostimulation includes one or more first stimulation parameters and one or more second stimulation parameters. The one or more first stimulation parameters can each be identified by the simulation to have a fixed value (or value range) with which a sensitivity to values of each of the one or more second stimulation parameters on the objective function is identified by the simulation to be minimized. The optimization can include identifying the one or more first stimulation parameters from the multiple stimulation parameters of the optimal pattern of neurostimulation and determining the fixed value for each parameter of the identified one or more first stimulation parameters using the computer simulation with the objective function and the model. The optimization can include selecting the optimal pattern of neurostimulation from multiple received patterns of neurostimulation or adjusting a received pattern of neurostimulation.

At 1895, a stimulation device is programmed to deliver the neurostimulation to the patient according to the optimal pattern of neurostimulation. Values of the one or more second parameters can be determined by value sweeping during the delivery of the neurostimulation.

FIG. 19 illustrates an embodiment of a pattern optimization circuit that can be configured (e.g., programmed) to perform steps 1891-1894 of method 1463. Pattern optimization circuit 1980 can be an example of pattern optimization circuit 1580 and can include an objective function input 1982, a model input 1984, a pattern input 1988, and optimization processing circuitry 1986. In various embodiments, pattern optimization circuit 1980 can be implemented in external programming device 802, such as using stimulation programming circuit 820 that is implemented in CP 630 and/or a device communicatively coupled to CP 630, depending on design considerations. Step 1895 can also be performed using external programming device 802. For example, programming control circuit 816 can generate information for programming the stimulation device to control the delivery of the neurostimulation according to the optimal pattern of neurostimulation.

Objective function input 1982 can to receive the objective function. Model input 1984 can receive the model. Pattern input 1988 can receive the one or more patterns of neurostimulation each including multiple stimulation parameters. Optimization processing circuitry 1986 can determine an optimal pattern of neurostimulation using simulation with the objective function, the model, and the one or more patterns of neurostimulation. The optimal pattern of neurostimulation include one or more first stimulation parameters and one or more second stimulation parameters. The one or more first stimulation parameters can each be identified by the simulation to have a fixed value (or value range) with which a sensitivity to values of each of the one or more second stimulation parameters on the objective function is identified by the simulation to be minimized.

In various embodiments, the one or more patterns of neurostimulation received by pattern input 1988 can include multiple patterns of neurostimulation, and optimization processing circuitry 1986 can determine the optimal pattern of neurostimulation by selecting a pattern of neurostimulation from the multiple patterns of neurostimulation. In some embodiments, optimization processing circuitry 1986 can further optimizing the selected pattern of neurostimulation by tuning it using the objective function, the model, and other patient-specific information. In other embodiments, the one or more patterns of neurostimulation received by pattern input 1988 includes a single pattern of neurostimulation, and optimization processing circuitry 1986 can determine the optimal pattern of neurostimulation by adjusting parameters of the single pattern of neurostimulation using the objective function, the model, and optionally other patient-specific information.

In various embodiments, the one or more patterns of neurostimulation received by pattern input 1988 can include one or more tonic pattern of neurostimulation (e.g., a train of neurostimulation pulses of identical parameters defining the pulse waveform and delivered at a constant pulse frequency) and/or one or more dynamic patterns of neurostimulation. A dynamic pattern of neurostimulation includes multiple stimulation parameters with at least one stimulation parameter being modulated by a modulating waveform. Examples of the modulated stimulation parameters include pulse amplitude, pulse width, pulse frequency, pulse shape, percentage of modulation, frequency of modulation, and shape of the modulating waveform.

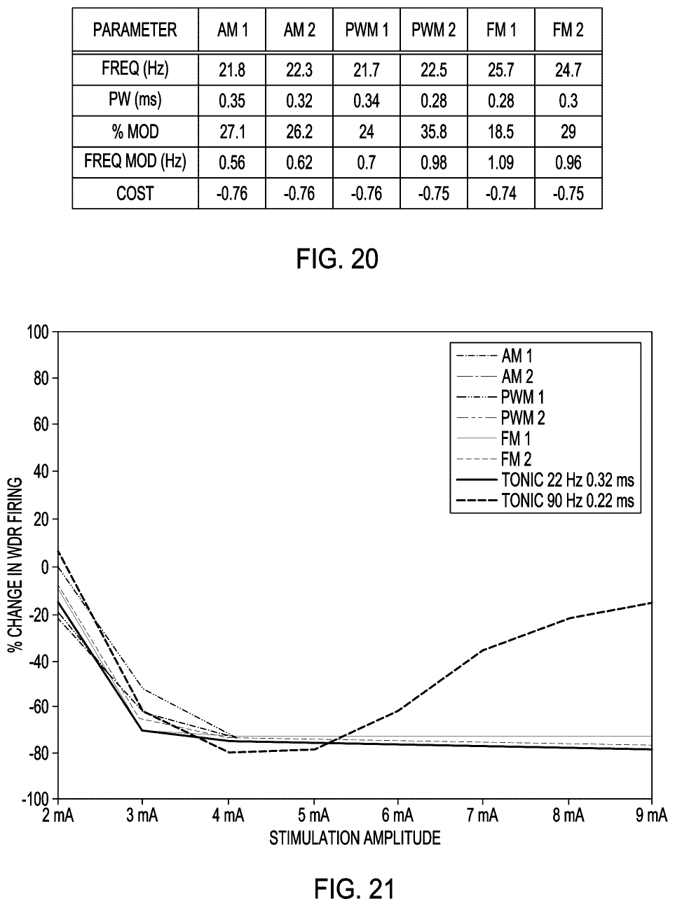

FIG. 20 illustrates an example of results of performing step 1894 of method 1890 using a simulation presented as a table. FIG. 21 illustrates another example of results of performing step 1894 of method 1890 using the simulation. The simulation was designed to demonstrate the method as applied to pain control using SCS and provide patterns of neurostimulation with specific values of identified first stimulation parameters. Parameters or labels shown in FIGS. 20-21 include:
Freq (Hz): pulse frequency in hertz;
PW (ms): pulse width in milliseconds;
% Mod: percentage of modulation;
Freq Mod (Hz): frequency at which the stimulation parameter is modulated (“envelope frequency”);
Cost: a score being the final value of a cost function (here a pain score being a quantitative measure of pain);
AM 1: first pattern with pulse amplitude being modulated;
AM 2: second pattern with pulse amplitude being modulated;
PWM 1: first pattern with pulse width being modulated;
PWM 2: second pattern with pulse width being modulated;
FM 1: first pattern with pulse frequency being modulated;
FM 2: second pattern with pulse frequency being modulated;
Tonic 22 Hz 0.32 ms: tonic pattern of neurostimulation pulses with pulse frequency of 22 Hz and pulse width of 0.32 ms; and
Tonic 90 Hz 0.22 ms: tonic pattern of neurostimulation pulses with pulse frequency of 90 Hz and pulse width of 0.22 ms.

From optimization of dynamic patterns of neurostimulation, a family of patterns of neurostimulation which greatly reduced the pain score in a simulation of SCS using a personalized model is identified for a broad range of pulse amplitudes. As shown in FIGS. 20-21:
These patterns all had pulse frequencies around 22 Hz and pulse widths around 0.32 ms;
The optimized patterns each had a single stimulation parameter being modulated by a sinusoidal wave with a percentage of modulation up to 36% and an envelope frequency between 0.5 and 1.1 Hz.
   Additional simulations showed that tonic stimulation with the stimulation frequency of 22 Hz and the pulse width of 0.32 ms resulted in comparable pain relief compared to the dynamic patterns of neurostimulation.

Results of the simulation show that the pain relief provided by these patterns of neurostimulation are likely comparable to the efficacy of an existing neurostimulation therapy program for a large range of stimulation amplitudes. It is concluded from the simulation that dynamic stimulation patterns with pulses frequencies between 20 and 30 Hz, pulse widths between 0.25 and 0.4 ms, modulation depth of pulse amplitude, pulse width, and/or pulse frequency between 0 and 40%, and modulation frequency between 0.5 and 1.1 Hz will result in substantial pain relief for a wide range of pulse amplitude (all the values of pulse amplitude used in the simulations). Therefore, dynamic patterns of neurostimulation with these parameter ranges can be used to determine amplitude and/or stimulation field, thereby reducing the need for determining values of multiple stimulation parameters.

Paresthesia generated by tonic stimulation at the pulse frequency of 22 Hz can be uncomfortable because the patient can feel each stimulation pulse. Dynamic stimulation has been shown to be associated with paresthesia that is better tolerated by patients. The simulation showed that SCS using dynamic stimulation patterns at 22 Hz can reduce pain score as well as tonic stimulation patterns at 22 Hz. Thus, combining the tonic stimulation at 22 Hz with dynamic stimulation at 22 Hz can make the stimulation waveform better tolerated by the patients while simultaneously providing the patients with pain relief. The simulation with the personalized model also predicts that sub-perception stimulation at 22 Hz can also be an effective therapy. In various embodiments, method 1890 can be performed to provide a viable alternative when programming is difficult or unsuccessful for an existing therapy approach, provide additional patterns of neurostimulation for effective pain management, require substantially less energy because the pulse frequency of 22 Hz is lower than many other patterns of neurostimulation, and/or provide one or more constant parameter values or values ranges for certain existing patterns of neurostimulation.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation to a patient using a stimulation device, the system comprising:
a personalization circuit including:
an assessment input configured to receive patient-specific information resulting from a patient survey, the patient-specific information including one or more patient dimensions of the neurostimulation;
a calibration input configured to receive calibration information resulting from a calibration, the calibration information including the patient's response to the neurostimulation; and
personalization processing circuitry configured to determine a personalized objective function using the one or more patient dimensions and to determine a personalized model having a model state personalized to the patient's response to the neurostimulation; and
a pattern optimization circuit including optimization processing circuitry configured to determine one or more optimal patterns of neurostimulation using the personalized objective function and the personalized model for programming the stimulation device to deliver the neurostimulation according to the one or more optimal patterns of neurostimulation.

2. The system of claim 1, wherein the personalization processing circuitry is configured to determine a personalized cost function as the personalized objective function, the personalized cost function being a quantitative measure of a condition of the patient expressed as a function of the one or more patient dimensions.

3. The system of claim 2, wherein the personalization processing circuitry is configured to optimize the personalized model by minimizing a difference between the patient's response to the neurostimulation as predicted by simulation using the personalized model and the received patient's response to the neurostimulation.

4. The system of claim 1, comprising an implantable stimulator as the stimulation device and an external system communicatively coupled to the implantable stimulator via telemetry, the external system including the personalization circuit and the pattern optimization circuit.

5. The system of claim 4, wherein the external system comprises an external programmer including the pattern optimization circuit and a remote device communicatively coupled to the external programmer via a telecommunication network and including the personalization circuit.

6. The system of claim 4, wherein the external system comprises an external programmer including the personalization circuit and the pattern optimization circuit.

7. The system of claim 4, wherein the external system is configured to perform the patient assessment including conducting a patient survey to determine the one or more patient dimensions of the neurostimulation.

8. The system of claim 4, wherein the external system is configured to perform the calibration to determine the patient's response to the neurostimulation by programming the implantable stimulator to deliver the neurostimulation to the patient and measuring one or more parameters representing the patient's response.

9. The system of claim 8, wherein the external system is configured to produce one or more response curves each being a measured parameter of the measured one or more parameters plotted as a function of a stimulation parameter used by the implantable stimulator to control the delivery of the neurostimulation.

10. A non-transitory computer-readable storage medium including instructions, which when executed by a system, cause the system to perform a method for delivering neurostimulation to a patient using a stimulation device, the method comprising:
receiving patient-specific information resulting from a patient survey, the patient-specific information including one or more patient dimensions of the neurostimulation;
receiving calibration information resulting from a calibration, the calibration information including the patient's response to the neurostimulation;
determining a personalized objective function using the received one or more patient dimensions;
determining a personalized model having a model state personalized to the patient's response to the neurostimulation;
determining one or more optimal patterns of neurostimulation using the personalized objective function and the personalized model; and
generating information for programming the stimulation device to deliver the neurostimulation according to the one or more optimal patterns of neurostimulation.

11. A method for delivering neurostimulation to a patient, the method comprising:
receiving patient-specific information resulting from a patient survey, the patient-specific information including one or more patient dimensions of the neurostimulation;
receiving calibration information resulting from a calibration, the calibration information including the patient's response to the neurostimulation;
determining a personalized objective function using the received one or more patient dimensions;
determining a personalized model having a model state personalized to the patient's response to the neurostimulation;
determining one or more optimal patterns of neurostimulation using the personalized objective function and the personalized model; and
programming a stimulation device to deliver the neurostimulation according to the one or more optimal patterns of neurostimulation.

12. The method of claim 11, wherein determining the personalized objective function comprises determining a personalized cost function being a quantitative measure of a condition of the patient expressed as a function of the one or more patient dimensions.

13. The method of claim 12, further comprising optimizing the personalized model by matching the patient's response to the neurostimulation as predicted by simulation using the personalized model to the received patient's response to the neurostimulation.

14. The method of claim 11, wherein determining the one or more optimal patterns of neurostimulation comprises selecting the one or more optimal patterns of neurostimulation from predefined patterns of neurostimulation.

15. The method of claim 11, wherein determining the personalized model comprises determining parameters of a predefined model.

16. The method of claim 11, further comprising conducting a patient survey to determine the one or more patient dimensions of the neurostimulation.

17. The method of claim 11, further comprising determining the patient's response to the neurostimulation by programming an implantable stimulator to deliver the neurostimulation to the patient and measuring one or more parameters representing the patient's response.

18. The method of claim 17, wherein determining the patient's response to the neurostimulation comprises producing one or more response curves each being a measured parameter of the measured one or more parameters plotted as a function of a stimulation parameter used by the implantable stimulator to control the delivery of the neurostimulation.

19. The method of claim 11, further comprising:
delivering the neurostimulation to the patient according to the one or more optimal patterns of neurostimulation for a prolonged time period;
receiving updated one or more patient dimensions during the prolong period according to a schedule;
adjusting the personalized objective function using the updated one or more patient dimensions;
adjusting the one or more optimal patterns of neurostimulation using the adjusted personalized objective function; and
programming the stimulation device to deliver the neurostimulation according to the adjusted one or more optimal patterns of neurostimulation.

20. The method of claim 19, further comprising:
receiving updated patient's response to the neurostimulation;
adjusting the personalized model to the updated patient's response to the neurostimulation;
adjusting the one or more optimal patterns of neurostimulation using the adjusted personalized model; and
programming the stimulation device to deliver the neurostimulation according to the adjusted one or more optimal patterns of neurostimulation.
